(19) United States
(12) Patent Application Publication
(10) Pub. No.: US 2023/0000854 A1
(43) Pub. Date: Jan. 5, 2023

(54) Application of substituted crotonamide

(71) Applicants: SUZHONG PHARMACEUTICAL GROUP CO., LTD., Taizhou, Jiangsu (CN); Jiangsu Suzhong Pharmaceutical Research Institute CO., LTD., Nanjing (CN)

(72) Inventors: Haitao TANG, Nanjing (CN); Haitao GE, Nanjing (CN); Nianhong YI, Nanjing (CN); Yuqiang ZHANG, Nanjing (CN); Sumin CAO, Nanjing (CN)

(21) Appl. No.: 17/780,516

(22) PCT Filed: Nov. 26, 2020
(86) PCT No.: PCT/CN2020/131638

§ 371 Date: May 26, 2022

(30) Foreign Application Priority Data
Nov. 27, 2019 (CN) 201911179729.9

(51) Int. Cl. A61K 31/4706

(52) U.S. Cl. CPC A61K 31/4706

(57) ABSTRACT

An application of a substituted crotonamide, in particular being an application of (E)-N-(3-cyano-7-ethoxyl-4-(3-ethynylphenylamino)quinoline-6-yl)-4-(dimethylamino)but-2-enamide and a pharmaceutically acceptable salt and solvate thereof in the preparation of a drug for treating cancer mediated by a rare EGFR mutation.

### FIELD OF THE INVENTION

This application relates to the field of medical technology, in particular to an application of substituted butenamide.

### BACKGROUND OF THE INVENTION

Non-small cell lung cancer (NSCLC) is a malignant tumor that seriously threatens human health. Despite the continuous improvement of surgery and chemotherapy techniques, the prognosis of patients is still poor, with a 5-year survival rate of less than 20%. At present, molecular targeted therapy targeting human epithelium growth factor receptor (EGFR) has become the most important way to treat NSCLC.

EGFR is the expression product of the proto-oncogene C-erbB-1, which is located on chromosome 7 and belongs to the transmembrane receptor tyrosine kinase. After EGFR binds to its ligand, it can activate downstream signaling pathways, regulate tumor cell proliferation, differentiation, angiogenesis, and inhibition of apoptosis, thereby regulating a series of tumor biological behaviors.

EGFR is the expression product of the proto-oncogene C-erbB-1, which is located on chromosome 7 and belongs to the transmembrane receptor tyrosine kinase. After EGFR binds to its ligand, it can activate downstream signaling pathways, regulate tumor cell proliferation, differentiation, angiogenesis, and inhibition of apoptosis, thereby regulating a series of tumor biological behaviors.

The currently used clinically targeted drugs against EGFR are EGFR tyrosine kinase inhibitors (EGFR-TKI). EGFR-TKI blocks the EGFR signaling pathway by inhibiting EGFR autophosphorylation, thereby inhibiting tumor cell proliferation and differentiation. Targeted therapy.

EGFR mutations can occur anywhere in the EGFR sequence. Generally, EGFR mutant strains are derived from mutations in the kinase domain (i.e., exons 18-24 in the EGFR sequence) or the extracellular domain (i.e., exons 2-16 in the EGFR sequence). One or more point mutations in exon 18 include L688P, V689M, P694L/S, N700D, L703V, E709K/Q/A/G/V, I715S, L718P, G719C/A/S/R or S720P/F. Deletions in exon 19 include delG719, delE746_E749, delE746_A750, delE746_A750insRP, delE746_A750insQP, delE746_T751, delE746_T751insA/I/V, delE746_T751insVA, delE746_S752, delE746_EinsVA, delE746_S752, delE746_S752insA/L/L747_Eins750, delL747_E747T747, delL747_AinsRP, delE746_A750insRP, delE746_T751 Q, delL747_T751insPI, delL747_S752, delL747_S752insQ, delL747_P753, delL747_P753insS/Q, delL747_L754insSR, delE749_A750, delE749_A750insRP, delE749_T751,delT751_I759, delT751_I759insS/N or delS752insS/N. Replication in exon 19 includes K739_I44dupKIPVAI. Point mutations in exon 19 include L730F, W731Stop, P733L, G735S, V742A, E746V/K, A750P, T751I, S752Y, P753S, A754P, or D761Y. In-frame inserts in exon 20 include D761_E762insEAFQ, A767_S768insTLA, V769_D770insY, V769_D770insCV, V769_D770insASV, D770_N771insD/G, D770_N771insNPG, D770_N771insSVQ, P772_H773_insN/CinsH773_VinsH773_Vins, P772_H773_N771insSVQ, P772_H773_N771insSVQ, P772_H773_VinsN/Vins. Deletions in exon 20 include delM766_A767, delM766_A767insAI, delA767_V769, delD770, or dellP772_H773insNP. Replication in exon 20 includes S768_D770dupSVD, A767_V769dupASV, or H773dupH. Point mutations in exon 20 include D761N, A763V, V765A/M, S768I, V769L/M, S768I, P772R, N771T, H773R/Y/L, V774M, R776G/H/C, G779S/F, T783A, T784F , L792P, L798H/F, T790M, R803W, K806E or L814P). Point mutations in exon 21 include G810S, N826S, L833V, H835L, L838V, A839T, K846R, T847I, H850N, V8511/A, I853T, L858M/R, A859T, L861Q/R, G863D, A864T, E866K, or G873E.

The efficacy of EGFR-TKI is closely related to the mutation status of EGFR gene. EGFR gene mutations are mainly concentrated in exons 18-21, including sensitive mutations and drug-resistant mutations.

Among Chinese lung cancer patients, the mutation rate of EGFR accounts for 30-50%. Among them, the mutation rate of exon 19 and 21 (L858R, L861I) accounts for about 90% of the total mutation rate, and the mutation rate of exon 18 accounts for 5 of the total mutation rate. The T790M mutation in exon 20 accounts for about 5% of the mutation rate. Among them, the deletion of exon 19 and the L858R mutation are called rare mutations, such as L861Q, G719X, S768I and so on.

CN102625797A discloses (E)-N-(3-cyano-7-ethoxy-4-(3-ethynylphenylamino)quinolin-6-yl)-4-(dimethylamino)butyl- The effect of 2-enamide on common EGFR mutants such as EGFR mutant L858R/T790M and EGFR mutant E746-A750, but the effect on rare mutations such as L861Q, G719X, S768I, etc. is not recorded.

It is clear that there is a need for new methods to inhibit cells with rare EGFR mutations (such as L861Q, G719X, S768I), and new therapies to treat cancers associated with this mutation will have far-reaching benefits.

### SUMMARY OF THE INVENTION

The purpose of the present invention is to provide an application of substituted butenamide, specifically to provide compound (E)-N-(3-cyano-7-ethoxyl-4-(3-ethynylphenylamino)quinoline-6-yl)-4-(dimethylamino)but-2-enamide, and/or its pharmaceutically acceptable salts, and/or solvent compounds.

The compound of the present invention (E)-N-(3-cyano-7-ethoxy-4-(3-ethynylphenylamino)quinolin-6-yl)-4-(dimethylamino) But-2-enamide and its pharmaceutically acceptable salts and solvent compounds have good inhibitory activity against cancers mediated by rare EGFR mutations such as L861Q, G719X, and 5768I.

The compound (E)-N-(3-cyano-7-ethoxy-4-(3-ethynylphenylamino)quinolin-6-yl)-4-(dimethylamino)butan- The structural formula of 2-enamide is shown in the following formula:

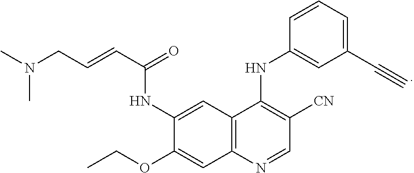 [SMILES: C#Cc1cccc(Nc2c(C#N)cnc3cc(OCC)c(NC(=O)/C=C/CN(C)C)cc23)c1]

The compound of the present invention (E)-N-(3-cyano-7-ethoxy-4-(3-ethynylphenylamino)quinolin-6-yl)-4-(dimethylamino) But-2-enamide and its pharmaceutically acceptable salts and solvent compounds can inhibit the proliferation of cells expressing EGFR mutant strains.

Further, the present invention provides (E)-N-(3-cyano-7-ethoxy-4-(3-ethynylphenylamino)quinolin-6-yl)-4-(dimethylamino) Application of but-2-enamide and its pharmaceutically acceptable salts and solvent compounds in the preparation of drugs for the treatment of rare EGFR mutation-mediated cancers.

Another aspect of the present invention is to provide a method for administering a therapeutically effective amount of (E)-N-(3-cyano-7-ethoxy-4-(3-acetylene) to a subject in need Phenylamino)quinolin-6-yl)-4-(dimethylamino)but-2-enamide and its pharmaceutically acceptable salts and solvent compounds to treat or alleviate rare EGFR mutations in subjects to guide the symptoms of cancer.

The rare EGFR mutations of the present invention are mutations other than EGFR19 exon deletion and L858R mutation, including any one or a combination of EGFR mutants L861Q, G719X, and S768I, which can be sensitive mutations (non-drug resistant) Mutation) or resistance mutation.

The “EGFR mutation-mediated cancer” in the present invention refers to a tumor characterized by EGFR gene mutations (including the specific mutations mentioned herein) that alter the biological activity of EGFR nucleic acid molecules or polypeptides. Tumors mediated by EGFR mutations can appear in any tissue, including brain, blood, connective tissue, liver, mouth, muscle, spleen, stomach, testes and trachea. Cancers mediated by EGFR mutations include non-small cell lung cancer (NSCLS), including one or more squamous cell carcinoma, adenocarcinoma, adenocarcinoma, bronchioloalveolar carcinoma (BAC), focally invasive BAC, and glands with BAC characteristics Cancer, and large cell carcinoma; nerve tumors, such as glioblastoma; pancreatic cancer; head and neck cancer (e.g., squamous cell carcinoma); breast cancer; colorectal cancer; epithelial cancer, including squamous cell carcinoma; ovarian cancer; Prostate cancer; adenocarcinoma; and including EGFR-mediated cancers.

The EGFR mutation-mediated cancer of the present invention is further non-small cell lung cancer.

In the present invention, “inhibiting the proliferation of cells expressing EGFR mutant strains” refers to moderately slowing, stopping or reversing the growth rate of cells expressing EGFR in vitro or in vivo. Ideally, when a suitable method for measuring cell growth rate is used for the measurement (for example, the cell growth assay described herein), the growth rate is slowed by at least 10%, 20%, 30%, 50%, or even 70%. The EGFR mutant strain can be any of the EGFR mutant strains described herein.

The pharmaceutically acceptable salts of the present invention include salts that are commonly used to form alkali metal salts and form addition salts of free acids or free bases approved by regulatory agencies. Salts are formed through ionic association, charge-charge interaction, covalent bonding, complexation, coordination, etc. As long as the salt is pharmaceutically acceptable, its nature is not critical.

The types of the pharmaceutically acceptable salts include, but are not limited to, acid addition salts formed by reacting the free base form of the compound with the following pharmaceutically acceptable acids: inorganic acids, such as hydrochloric acid, hydrobromic acid, sulfuric acid, phosphoric acid Etc.; or organic acids, such as acetic acid, propionic acid, glycolic acid, pyruvic acid, lactic acid, malonic acid, succinic acid, malic acid, maleic acid, fumaric acid, tartaric acid, citric acid, methanesulfonic acid, benzenesulfonic acid, toluenesulfonic acid, gluconic acid, glutamic acid, hydroxynaphthoic acid, salicylic acid, etc.

Other examples of such salts can be found in Berge et al., J. Pharm. Sci, 66, 1 (1977). In some embodiments, conventional methods are used to form the salt. For example, the phosphoric acid of the compound of the present invention is prepared by mixing the free base of the desired compound in the desired solvent or combination of solvents with the desired stoichiometric amount of phosphoric acid at a desired temperature, usually under heating (depending on the boiling point of the solvent) salt. In one embodiment, the salt precipitates and crystallizes (i.e. if it has crystalline properties) after cooling (slowly or rapidly). In addition, this document also includes half-salt, mono-salt, di-salt, tri-salt, and multiple-salt forms of the compounds of the present invention. Similarly, this document also includes the compound, its salt or hemihydrate, monohydrate, dihydrate, trihydrate, and polyhydrate forms.

In some embodiments, the compound is hydrochloride, hydrobromide, sulfate, phosphate or metaphosphate, acetate, propionate, caproate, cyclopentane propionate, glycolic acid Salt, pyruvate, lactate, malonate, succinate, malate, maleate, fumarate, trifluoroacetate, tartrate, citrate, benzoate , 3-(4-hydroxybenzoyl)benzoate, cinnamate, mandelate, methanesulfonate, ethanesulfonate, 1,2-ethanedisulfonate, 2-hydroxyethanesulfonic acid Salt, benzenesulfonate, toluenesulfonate, 2-naphthalenesulfonate, 4-methylbicyclo-[2.2.2]oct-2-ene-1-carboxylate, glucoheptonate, 4 ′4-methylene bis-(3-hydroxy-2-ene-1-carboxylic acid) salt, 3-phenylpropionate, trimethyl acetate, tert-butyl acetate, lauryl sulfate , gluconate, glutamate, naphthoate, salicylate, stearate, muconate, butyrate, phenylacetate, phenylbutyrate, valproate acid salts and so on. In a preferred embodiment, the compound is hydrochloride, benzenesulfonate, methanesulfonate, maleate, or a hydrate thereof, such as a monohydrate.

The compound of the present invention (E)-N-(3-cyano-7-ethoxy-4-(3-ethynylphenylamino)quinolin-6-yl)-4-(dimethylamino)) When the but-2-enamide and its pharmaceutically acceptable salts and solvent compounds are administered to a patient, they form a pharmaceutical composition with pharmaceutically acceptable excipients, and are formulated into a suitable pharmaceutical preparation form for administration.

In the pharmaceutical composition used in the present invention, the pharmaceutically acceptable excipients include carriers, excipients, binders, fillers, suspending agents, fragrances, sweeteners, disintegrants, dispersants, and surface agents. Active agents, lubricants, colorants, diluents, solubilizers, wetting agents, plasticizers, stabilizers, penetration enhancers, wetting agents, defoamers, antioxidants, preservatives, or one or more of them combination. The pharmaceutical composition facilitates the administration of the compound to the organism. In practicing the treatments or methods of use provided herein, a therapeutically effective amount of the compound described herein is administered in the form of a pharmaceutical composition to a mammal suffering from the disease, disorder, or condition to be treated. In some embodiments, the mammal is a human. The therapeutically effective amount can vary widely depending on the severity of the disease, the age and relative health of the individual, the potency of the compound used, and other factors. The compounds can be used alone or as a component of a mixture in combination with one or more therapeutic agents.

In the pharmaceutical composition used in the present invention, the pharmaceutically acceptable excipients include carriers, excipients, binders, fillers, suspending agents, fragrances, sweeteners, disintegrants, dispersants, and surface agents. Active agents, lubricants, colorants, diluents, solubilizers, wetting agents, plasticizers, stabilizers, penetration enhancers, wetting agents, defoamers, antioxidants, preservatives, or one or more of them combination. The pharmaceutical composition facilitates the administration of the compound to the organism. In practicing the treatments or methods of use provided herein, a therapeutically effective amount of the compound described herein is administered in the form of a pharmaceutical composition to a mammal suffering from the disease, disorder, or condition to be treated. In some embodiments, the mammal is a human. The therapeutically effective amount can vary widely depending on the severity of the disease, the age and relative health of the individual, the potency of the compound used, and other factors. The compounds can be used alone or as a component of a mixture in combination with one or more therapeutic agents.

The pharmaceutical preparations of the present invention include, but are not limited to, aqueous liquid dispersions, self-emulsifying dispersions, solid solutions, liposome dispersions, aerosols, solid dosage forms, powders, immediate release formulations, controlled release formulations, disintegration (fastmelt) formulations, tablets, capsules, pills, delayed release formulations, extended release formulations, pulsatile release formulations, multiparticulate formulations, and mixed rapid release and controlled release formulations.

The amount of the compound administered by the patient and the dosage regimen for treating cancer with the compound and/or composition of the present invention depends on a variety of factors, including the age, weight, sex and medical condition of the individual, the type of disease, the severity of the disease, and the administration. The pathway and frequency and the specific compound used. Therefore, the dosage regimen can vary widely, but can be determined routinely using standard methods. In some embodiments, a daily dose of about 0.01-500 mg/kg body weight, advantageously a daily dose of about 0.01-50 mg/kg body weight, more advantageously a daily dose of about 0.01 to about 30 mg/kg body weight, more advantageously about 0.1 A daily dose of −10 mg/kg body weight and even more advantageously a daily dose of about 0.5-3 mg/kg body weight is appropriate and should be available for all methods of use disclosed herein. The daily dose can be administered in 1 to 4 doses per day. In some embodiments, the effective therapeutic dose administered to the patient is 50-250 mg, preferably 100 mg once a day for 28 consecutive days.

Suitable routes for patient administration include, but are not limited to, oral, intravenous, rectal, aerosol, parenteral, ocular, pulmonary, transmucosal, transdermal, vaginal, aural, nasal and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

The compound of the present invention (E)-N-(3-cyano-7-ethoxy-4-(3-ethynylphenylamino)quinolin-6-yl)-4-(dimethylamino)) But-2-enamide and its pharmaceutically acceptable salts and solvent compounds have outstanding therapeutic effects on rare EGFR mutation-mediated cancers. Compared with general compounds, they are inferior to unusual EGFR mutation-mediated cancers. Cancers mediated by EGFR mutations are different, and the compounds of the present invention have better therapeutic effects than non-rare EGRR mutations such as T790M or L858R/T790M mutations.

The compound of the present invention (E)-N-(3-cyano-7-ethoxy-4-(3-ethynylphenylamino)quinolin-6-yl)-4-(dimethylamino)) But-2-enamide and its pharmaceutically acceptable salts and solvent compounds have outstanding therapeutic effects on rare EGFR mutation-mediated cancers. Compared with general compounds, they are inferior to unusual EGFR mutation-mediated cancers. Cancers mediated by EGFR mutations are different, and the compounds of the present invention have better therapeutic effects than non-rare EGRR mutations such as T790M or L858R/T790M mutations.

### DETAILED DESCRIPTION

The following examples are used to further describe the present invention, but these examples do not limit the scope of the present invention.

(E)-N-(3-cyano-7-ethoxy-4-(3-ethynylphenylamino)quinolin-6-yl)-4-(dimethylamino) involved in the examples of the application) But-2-enamide was prepared according to the method described in WO2010151710, (E)-N-(3-cyano-7-ethoxy-4-(3-ethynylphenylamino)quinolin-6-yl)-4-(dimethylamino)but-2-enamide maleate monohydrate was prepared according to the method described in CN104513200A, and it was also named (E)-N-{4-[(3-ethynyl Anilino)-3-cyano-7-ethoxy-6-quinolinyl]}-4-(dimethylamino)-2-butenamide maleate.

### Example 1 IC50 Determination of Protein Kinase

The compound tested in this example is (E)-N-(3-cyano-7-ethoxy-4-(3-ethynylphenylamino)quinolin-6-yl)-4-(dimethyl Amino)but-2-enamide.

Part 1. Experimental Design

The IC50 of the test compound against protein kinase, the test substance is set at 10 semi-logarithmic concentrations (from 1×10⁻⁰⁶ M to 3×10⁻¹¹ M), and the IC50 is determined in a single well.

Part 2. Test Substance

The test substance is two tubes of solid, and ProQinase is transferred to −20° C. for subsequent use.

Before the test, a storage solution of 1×10⁻⁰³ M test substance was prepared with 100% DMSO and further diluted to 1×10⁻⁰⁴ M/100% DMSO.

The test substance was diluted semi-logarithmically into a 96-well plate with 100% DMSO and prepared from 1×10⁻⁰⁴ M to 3×10⁻⁰⁹ M. Before use, the test substance is diluted 1:10 with water to obtain 1×10⁻⁰⁵ M to 3×10⁻¹⁰ M test substance samples containing 10% DMSO.

Add 5 μl of each concentration to the test (see Part 3 Protein Kinase Test), the final volume of the test is 50 μl. The test substance is measured at a concentration of 1×10⁻⁰6 M to 3×10⁻¹¹ M. The final concentration of DMSO in all reaction wells is 1%.

Part 3. Protein Kinase Test

Protein kinase test by radiometry (³³PanQinase® activity determination method) is used to determine the activity of 8 protein kinases. All experiments were performed in a 96-well plate of Perkin Elmer (Boston, Mass., USA) with a reaction volume of 50 μl. Reagents and samples are added in 4 steps:

10 μl non-radioactive ATP aqueous solution;

25 μl buffer/[γ⁻³³ P]-ATP mixture;

5 μl test substance containing 10% DMSO;

10 μl enzyme/substrate mix.

The substrate is Poly(Glu, Tyr) 4:1, code name SIG_20K5903.

All experiments include 70 mM HEPES-NaOH buffer at pH7.5, 3 mM MgCl₂, 3 mM MnC, 3 μM sodium orthovanadate, 1.2 mM DTT, ATP (concentration varied, concentration related to the apparent ATP-Km of each enzyme, see Table 1), [γ⁻³³P]-ATP (approximately 8×1005 cpm per well), protein kinase (concentration varied, see Table 1), and substrate (concentration varied, see Table 1).

The concentrations of enzymes and substrates used in the experiment are shown in Table 1:

TABLE 1
Concentrations of enzymes and substrates used

| | kinase | [kinase] | [kinase] | [ATP] | [substrate] |
|---|---|---|---|---|---|
| No. | name | ng/50 μL | nM* | μM | ng/50 μL |
| 1 | EGF-R G719C | 20 | 4.5 | 1.0 | 0.125 |
| 2 | EGF-R G719S | 10 | 2.2 | 0.3 | 0.125 |
| 3 | EGF-R L861Q | 25 | 5.6 | 0.3 | 0.125 |
| 4 | EGF-R 790M | 10 | 2.2 | 0.3 | 0.125 |
| 5 | EGF-R | 10 | 2.2 | 0.3 | 0.125 |
| | T790M/L858R | | | | |
| 6 | EGF-R wt | 5 | 1.1 | 0.3 | 0.125 |
| 7 | ERBB2 | 100 | 21.3 | 1.0 | 0.125 |
| 8 | ERBB4 | 5 | 1.0 | 0.3 | 0.125 |

The reaction system was incubated at 39° C. for 60 minutes. The reaction was terminated with 50 μl 2% (v/v) H₃PO₄, and the plate was washed twice with 200 μl 0.9% (w/v) NaCl. Microplate scintillation counter (Microbeta, Wallac) measures the enzyme activity-dependent transfer of ³³Pi (in “cpm”).

All experiments were done with Beckman Coulter Biomek 2000/SL robot.

Part 4. Data Analysis

The blank control places 3 reaction wells without adding enzyme, and taking the median of the cpm value of the 3 reaction wells as the reading for the blank. This value reflects the non-specific binding of radioactivity in the absence of protein kinase or substrate. In addition, set 3 reaction wells without adding test substance, and take the median of the cpm value of 3 reaction wells as a negative control, that is, the enzyme activity without any inhibitor. The difference between the negative control and the blank is the 100% activity of each enzyme.

As with the negative control, the blank value should be subtracted from the value of the corresponding 10 test substances. The residual enzyme activity of each well is calculated according to the following formula: residual enzyme activity (%)=100×[(test substance cpm value-blank control)/(negative control-blank control)].

Each test substance is set to 10 concentrations to act on each enzyme, so the data analysis is based on these 10 residual enzyme activity values. Use Prism5.04 (Graphpad, San Diego, Calif., USA www.graphpad.com), fix the maximum value of 100%, the minimum value of 0%, and perform S-curve fitting to calculate IC50.

Part 5. Results

Table 2 lists the IC50 values of the tested compounds acting on 8 kinases.

TABLE 2
IC50 values of the tested compounds acting on 8 kinases

| No. | Kinase | IC50 (M) |
|---|---|---|
| 1 | EGF-R G719C | 1.40 × 10⁻⁰⁹ |
| 2 | EGF-R G719S | 1.50 × 10⁻⁰⁹ |
| 3 | EGF-R L861Q | 1.90 × 10⁻⁰⁹ |
| 4 | EGF-R 790M | 1.20 × 10⁻⁰⁷ |
| 5 | EGF-R | 8.80 × 10⁻⁰⁸ |
| | T790M/L858R | |
| 6 | EGF-R wt | 1.30 × 10⁻⁰⁹ |
| 7 | ERBB2 | 2.70 × 10⁻⁰⁸ |
| 8 | ERBB4 | 1.00 × 10⁻⁰⁸ |

The above data shows that the inhibitory effect of the tested compound on rare mutations is better than the general non-rare mutation inhibitory effect.

### Example 2 An Exploratory Clinical Research Trial for the Treatment of Locally Advanced or Metastatic Non-Small Cell Lung Cancer

(I) Subject selection criteria

1. Inclusion criteria:

(1) Age from 18 to 75 (including 18 and 75) years old, regardless of gender.

(2) Patients with locally advanced or metastatic NSCLC confirmed by histopathology and/or cytology.

(3) The patient's EGFR has one or more of L861Q, G719X, and S768I mutations, and does not have T790M mutation, exon 19 deletion mutation, exon 20 insertion mutation, or L858R mutation.

(4) The ECOG physical strength score is 0, 1, or 2.

(5) Expected survival time>3 months.

(6) According to Version 1.1 of the Curative Effect Evaluation Criteria for Solid Tumors (RECIST), there is at least one evaluable tumor lesion.

(7) Sufficient blood system function, liver function, kidney function and blood coagulation function: Absolute neutrophil count≥1.5×109/L, platelet count≥90×109/L, hemoglobin≥90g/L; Total bilirubin≤1.5×upper limit of normal (ULN), alanine aminotransferase (ALT)≤2.5×ULN, aspartate aminotransferase (AST)≤2.5×ULN (total bilirubin in patients with liver metastasis≤3.0×ULN , ALT≤5.0×ULN, AST≤5.0×ULN); Creatinine≤1.0×ULN, or creatinine clearance≥60mL/min (using Cockcroft-Gault method); International normalized ratio (INR)≤1.5.

(8) Eligible patients (male and female) with fertility must agree to use reliable contraceptive methods (hormonal or barrier method or abstinence) during the trial period and at least 90 days after the last medication; female patients of childbearing age within 7 days before enrollment The blood human chorionic gonadotropin (HCG) pregnancy test must be negative; male patients cannot do sperm donation within 90 days after the first dose to the last dose.

(9) All patients must be informed of this study before beginning to accept any inspections prescribed by this trial, and voluntarily sign a written informed consent (ICF) approved by the ethics committee.

2. Exclusion criteria:

(1) Have received any epidermal growth factor receptor tyrosine kinase inhibitor (EGFR-TKI) anti-tumor therapy before enrollment.

(2) Patients who have received chemotherapy, anti-angiogenesis and other systemic anti-tumor treatments in the past require a washout period of 4 weeks or more; those who have received anti-PD-1/PD-L1 immunotherapy in the past and have not experienced immune pneumonia For patients, a washout period of 4 weeks or more is required; for patients who have previously received radiotherapy or anti-tumor Chinese patent medicine for non-target lesions for the purpose of relieving symptoms, a washout period of 2 weeks or more is required.

(3) Major organ surgery (excluding needle biopsy) or significant trauma occurred within 4 weeks before enrollment.

(4) At the time of screening, the adverse reaction (ADR) of any previous treatment has not yet recovered to the Common Terminology Criteria for Adverse Events (CTCAE) 5.0 grade evaluation≤grade 1 (except for hair loss).

(5) Inability to take drugs orally, severe chronic gastrointestinal dysfunction (CTCAE 5.0 rating≥3), malabsorption syndrome or any other conditions that affect the absorption of the gastrointestinal tract (for example: peptic ulcer, intestinal tract) Obstruction, irritable bowel syndrome, Crohn's disease, gastroesophageal reflux disease, etc.).

(6) Have immunodeficiency, including but not limited to human immunodeficiency virus (HIV) antibody test (enzyme-linked immunoassay or Western spot method) positive, and active rheumatic immune disease.

(7) There is unstable central nervous system metastasis or meningeal metastasis with clinical symptoms, or there is other evidence that the patient's central nervous system metastasis or meningeal metastasis has not been controlled, and the investigator judges that it is not suitable for inclusion; clinical symptoms suspect that there is brain Or patients with leptomeningeal disease need to be excluded by computed tomography/magnetic resonance imaging (CT/MRI).

(8) A history of severe bullous or exfoliative skin diseases (CTCAE 5.0 grade evaluation≥grade 3).

(9) During screening, there are uncontrolled active infections (such as syphilis, HIV, hepatitis B virus (HBsAg positive, HBV-DNA>1000 cps/ml and AST/ALT>2.0 xULN) or hepatitis C virus (HCV) infection.

(10) A history of severe cardiovascular disease [New York College of Cardiology (NYHA) Heart Function Grade III or IV], including but not limited to ventricular arrhythmia requiring clinical intervention; within 6 months before enrollment Acute coronary syndrome, congestive heart failure, stroke, or other cardiovascular events of grade III and above; at the time of screening, NYHA cardiac function grade≥grade II or left ventricular ejection fraction (LVEF)<50%.

(11) There is a history of other serious systemic diseases (CTCAE 5.0 grade evaluation≥grade 3), and the investigator judges it is not suitable for participating in clinical trials.

(12) Participated in other clinical trials within 4 weeks before enrollment.

(13) Known alcohol or drug dependence.

(14) Patients with mental disorders or researchers who believe that poor compliance are not suitable for participating in the study.

(15) Women during pregnancy or lactation.

(16) Known to be allergic to the active ingredients or excipients of the test drug.

(17) Long-term treatment with adrenal steroids is required (equivalent to the daily dose of prednisolone≥20 mg).

(18) Suffered from other malignant tumors in the past 5 years or at the same time (except for cured skin basal cell carcinoma and cervical carcinoma in situ; except for those without recurrence>3 years after radical mastectomy).

(II). Administration method and dosage

(E)-N-(3-cyano-7-ethoxy-4-(3-ethynylphenylamino)quinolin-6-yl)-4-(dimethylamino)but-2-ene Amide maleate monohydrate capsules (refer to Chinese patent application 201911180660.1), taken orally at least 1 hour before meals or 2 hours after meals; 100 mg once a day for 28 days. Every four weeks since the start of administration is a cycle.

(III). Therapeutic effect

TABLE 3
The sum of the longest diameters of the target lesions at
the end of the treatment cycle

| | | | Sum of the longest | | | |
|---|---|---|---|---|---|---|
| Patient | | Mutation | Diameters of all lesions | | | |
| No. | Gender | Type | Selection cycle | 2^{nd} cycle | 4^{th} cycle | Changes % |
| 001 | Female | L861Q | 65.8 mm | 35 mm | 30.1 mm | −54% |
| 002 | Female | L861Q | 58.6 mm | 40.4 mm | 36.7 mm | −37% |
| 003 | Female | G719X | 20.8 mm | 16 mm | 13.6 mm | −35% |
| 004 | Female | G719X, S768I | 105.0 mm | 48.0 mm | 43.0 mm | −59% |
| 005 | Male | S768I | 62.8 mm | 38.7 mm | 39.6 mm | −37% |
| 006 | Male | G719X | 81.0 mm | 31.0 mm | 34.8 mm | −57% |
| 007 | Male | S768I | 89.1 mm | 54.8 mm | 57.3 mm | −38% |
| 008 | Female | G719X | 23.9 mm | 14.7 mm | 18.2 mm | −38% |
| 009 | Female | L861Q, G719X | 88.4 mm | 51.6 mm | 47.9 mm | −46% |
| 010 | Female | L861Q | 18.0 mm | 8.9 mm | mm | −51% |

TABLE 4
The sum of the shortest diameters of the target lesions in the treatment cycle

| | | | Sum of the longest | Sum of shortest | | |
|---|---|---|---|---|---|---|
| Patient | | Mutation | Diameters of all | Diameters of all | | PFS |
| No. | Gender | Type | lesions | lesions | Evaluation | (month) |
| 001 | Female | L861Q | 65.8 mm | 29.9 mm | PR | 8 |
| 002 | Female | L861Q | 58.6 mm | 36.7 mm | PR | 14 |
| 003 | Female | G719X | 20.8 mm | 13.6 mm | PR | 14 |
| 004 | Female | G719X, S768I | 105.0 mm | 41.0 mm | PR | 13 |
| 005 | Male | S768I | 62.8 mm | 38.7 mm | PR | 10 |
| 006 | Male | G719X | 81.0 mm | 27.0 mm | PR | 12 |
| 007 | Male | S768I | 89.1 mm | 54.8 mm | PR | 12 |
| 008 | Female | G719X | 23.9 mm | 13.5 mm | PR | 11 |
| 009 | Female | L861Q, G719X | 88.4 mm | 47.3 mm | PR | 9 |

PFS: progression-free survival

The above treatment results show that cancer patients with rare EGFR mutation-mediated cancers have shown good therapeutic effects after treatment.

What is claimed is:

1. An application of (E)-N-(3-cyano-7-ethoxyl-4-(3-ethynylphenylamino)quinoline-6-yl)-4-(dimethylamino)but-2-enamide, or a pharmaceutically acceptable salt, or solvate thereof in the preparation of a drug for treating cancer mediated by a rare EGFR mutation.

2. The application according to claim 1, wherein the pharmaceutically acceptable salt is hydrochloride salt, benzenesulfonate salt, methanesulfonate salt, or maleate salt.

3. The application according to claim 1, wherein the pharmaceutically acceptable salt is a hydrate, preferably a hemihydrate or a monohydrate.

4. The application according to claim 1, wherein the rare EGFR mutations include but are not limited to any one or a combination of EGFR mutants: L861Q, G719X, and/or S768I.

5. The application according to claim 1, wherein the cancer is non-small cell lung cancer.

6. A method for treating rare EGFR mutation-mediated cancer, which is characterized by administering a therapeutically effective amount of (E)-N-(3-cyano-7-ethoxyl-4-(3-ethynylphenylamino)quinoline-6-yl)-4-(dimethylamino)but-2-enamide, or a pharmaceutically acceptable salt, or solvent thereof.

7. The method according to claim 6, wherein the therapeutically effective amount is from 50 to 250 mg per day, preferably 100 mg a day.

8. The method according to claim 6, wherein the therapeutically effective amount is daily dose/body weight from 0.01 to 500 mg/kg, preferably from 0.01 to 50 mg/kg, more preferably from 0.01 to 30 mg/kg, further from 0.1 to 10 mg/kg or from 0.5 to 3mg/kg.

9. The method according to claim 6, wherein the rare EGFR mutations include but are not limited to EGFR mutants: L861Q, G719X, and/or S768I.

10. The method according to claim 6, wherein the pharmaceutically acceptable salt is hydrochloride salt, benzenesulfonate salt, methanesulfonate salt, maleate salt, and more preferably hemihydrate or monohydrate of the pharmaceutically acceptable salt.
